(19) United States
(12) Patent Application Publication
(10) Pub. No.: US 2021/0008145 A1
(43) Pub. Date: Jan. 14, 2021

(54) EDICINE COMPOSITION FOR TREATMENT OF HUMAN PAPILLOMAVIRUS INFECTION AND PREPARATION METHOD THEREOF

(71) Applicant: Jiangsu Zhengyang Pharmaceutical Co., Ltd., Nanjing (CN)

(72) Inventor: Rucheng RONG, Nanjing (CN)

(21) Appl. No.: 17/037,617

(22) Filed: Sep. 29, 2020

Related U.S. Application Data
(63) Continuation-in-part of application No. PCT/CN2019/087286, filed on May 16, 2019.

(30) Foreign Application Priority Data
Jul. 12, 2018 (CN) 201810765903.7

(51) Int. Cl. A61K 36/90; A61K 36/54; A61K 36/704; A61K 9/06; A61K 9/02; A61P 31/12; A61P 31/04

(52) U.S. Cl. CPC A61K 36/90; A61K 36/54; A61K 36/704; A61K 9/06; A61K 2236/333; A61P 31/12; A61P 31/04; A61K 2236/331; A61K 9/02

(57) ABSTRACT

A pharmaceutical composition, including: 30-60 parts by weight of Smilax Glabra Root; 10-20 parts by weight of Cinnamon Twig; 10-20 parts by weight of Chinese Knotweed; and 10-20 parts by weight of Commelina Communis.

### CROSS-REFERENCE TO RELATED APPLICATIONS

This application is a continuation-in-part of International Patent Application No. PCT/CN2019/087286 with an international filing date of May 16, 2019, designating the United States, now pending, and further claims foreign priority benefits to Chinese Patent Application No. 201810765903.7 filed Jul. 12, 2018. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of traditional Chinese medicine, and more particularly, to a traditional Chinese medicine composition for treatment of Human papillomavirus (HPV) infection and a preparation method thereof.

### SUMMARY

It is an object of the disclosure to provide a traditional Chinese medicine composition for treatment of HPV infection and associated diseases including vaginitis, cervicitis, and cervical ectropion caused by HPV.

The traditional Chinese medicine composition comprises: 30-60 parts by weight of Smilax Glabra Root, 10-20 parts by weight of Cinnamon Twig, 10-20 parts by weight of Chinese Knotweed, and 10-20 parts by weight of Commelina Communis.

In a class of this embodiment, the composition comprises 30-55 parts by weight of Smilax Glabra Root, 12-18 parts by weight of Cinnamon Twig, 10-18 parts by weight of Chinese Knotweed and 12-18 parts by weight of Commelina Communis.

The disclosure also provides a pharmaceutical preparation comprising the traditional Chinese medicine composition.

The pharmaceutical preparation has a dosage form of creams, lotions, gels, or suppositories.

The pharmaceutical preparation is prepared by pulverizing the traditional Chinese medicine composition into decoction pieces, dissolving the decoction pieces in ethanol, obtaining an extract, concentrating and drying the extract at low temperature.

The pharmaceutical preparation is prepared by dissolving the traditional Chinese medicine composition in water, extracting, and refining by a macroporous resin.

The pharmaceutical preparation is prepared by dissolving the traditional Chinese medicine composition in ethanol, extracting, and refining by a macroporous resin.

A method for preparing the pharmaceutical preparation comprises: evenly mixing raw materials, dissolving the raw materials in a solvent, obtaining an extract, and concentrating the extract to obtain a pharmaceutical cream.

The solvent used for extraction is ethanol or water, and the effective ingredients of the extract are concentrated under reduced pressure.

Specifically, a method of preparing the pharmaceutical preparation comprises:

1) mixing 30-60 parts by weight of Smilax Glabra Root, 10-20 parts by weight of Cinnamon Twig, 10-20 parts by weight of Chinese Knotweed, and 10-20 parts by weight of Commelina Communis, thus yielding a mixture;

2) dissolving the mixture in a solvent, decocting the mixture dissolved in the solvent, thus yielding a first filtrate and a first residue;

3) collecting the first filtrate, dissolving the first residue in the solvent, and decocting the first residue dissolved in the solvent, thus yielding a second filtrate and a second residue;

4) repeating 3) for several times, mixing collected filtrates, and condensing the filtrates to yield an extract having a relative density of 1.10-1.40 at 60° C.

In a class of this embedment, the solvent is water.

In a class of this embedment, the solvent is an ethanol solution; the ethanol solution has a concentration of 50-80% (v/v), and a volume weight ratio of the ethanol solution to the mixture is 5-15.

In a class of this embedment, in 2), the mixture and the filtrates are decocted in the solvent at 50-75° C. for 2-5 hours.

In a class of this embedment, in 4), repeating 3) for 2-5 times, and the filtrates are condensed at 50-80° C.

Specifically, the preparation method comprises:

1) mixing and pulverizing the Smilax Glabra Root, Cinnamon Twig, Chinese Knotweed, and Commelina Communis into decoction pieces;

2) dissolving the decoction pieces in 60% ethanol, the volume of which is 10 folds of the mass of the decoction pieces, filtering the decoction pieces, collecting a first filtrate, dissolving a filter residue in 60% ethanol, the volume of which is 10 folds of the mass of the filter residue, filtering the filter residue, collecting a second filtrate, repeating operations of dissolving and filtering for 2-3 times with each dissolving for 1-2 hours, and mixing collected filtrates; and

3) concentrating the collected filtrates at 60° C. under reduced pressure to yield a pharmaceutical cream with a relative density of 1.10-1.40.

Specifically, the preparation method comprises the following steps of:

1) mixing and pulverizing the Smilax Glabra Root, Cinnamon Twig, Chinese Knotweed, and Commelina Communis into decoction pieces;

2) dissolving the decoction pieces in water, the volume of which is 12 folds of the mass of the decoction pieces, filtering the decoction pieces, collecting a first filtrate, dissolving a filter residue in water, the volume of which is 12 folds of the mass of the filter residue, filtering the filter residue, collecting a second filtrate, repeating operations of dissolving and filtering with each dissolving for 1-2 hours, and mixing collected filtrates;

3) adsorbing the collected filtrates in 2) on an AB-8 macroporous absorbent resin, eluting the resin consecutively with water and 75% ethanol, and colleting an eluent comprising 75% ethanol;

4) concentrating the eluent comprising 75% ethanol in 3) at 60° C. under reduced pressure to yield a pharmaceutical cream with a relative density of 1.10-1.40.

Specifically, the preparation method comprises the following steps of:

1) mixing and pulverizing the Smilax Glabra Root, Cinnamon Twig, Chinese Knotweed, and Commelina Communis into decoction pieces;

2) dissolving the decoction pieces with 60% ethanol, the volume of which is 10 folds of the mass of the decoction pieces, filtering the decoction pieces, collecting a first filtrate, repeating operations of dissolving and filtering for 2 times with each dissolving for 1-2 hours, and mixing collected filtrates;

3) adsorbing the collected filtrates in 2) on an AB-8 macroporous absorbent resin, eluting the resin consecutively with water and 75% ethanol, and colleting an eluent comprising 75% ethanol; and

4) concentrating the eluent comprising 75% ethanol in 3) at 60° C. under reduced pressure to yield a pharmaceutical cream with a relative density of 1.10-1.40.

In a class of this embodiment, the filtrates are obtained by reflux extraction and then mixed.

In a class of this embodiment, the pharmaceutical cream is mixed with an excipient to form a gel or a suppository.

In a class of this embodiment, the pharmaceutical cream is mixed with an excipient to form a lotion.

The disclosure provides a method for prevention and/or treatment of human papillomavirus (HPV) infection comprising administering a patient in need thereof the pharmaceutical preparation.

The disclosure provides a method for prevention and/or treatment of vaginitis, cervicitis, or cervical ectropion comprising administering a patient in need thereof the pharmaceutical preparation. Specifically, the pharmaceutical preparation is used for treatment of HPV infection and associated diseases including vaginitis, cervicitis, and cervical ectropion caused by HPV.

The advantages associated with the pharmaceutical preparation of the disclosure include but are not limited to fewer and mild side effects, convenient use, and safety over conventional drugs used to treat HPV infection and associated diseases, such as cervical intraepithelial neoplasia (CIN) 1 and CIN 2. The clinical trials show that the probability of success of the pharmaceutical preparation for treatment of HPV infection and associated diseases exceeds 85%.

The traditional Chinese medicine composition of the disclosure contains effective active ingredients with less toxic side effects, such as flavonoids and alkaloids. Studies have confirmed that the traditional Chinese medicinal composition is able to kill HPV and human cervical cancer HeLa cells, and to treat cervicitis, which is suitable for treatment of HPV infection and the associated diseases, such as vaginitis, cervicitis, and cervical ectropion. The dosage forms of the pharmaceutical preparation, including gel, suppository and lotion, are easy for patients to use and improve patient compliance with medication. The pharmaceutical preparation with the dosage forms can stay in the vagina for a long time and penetrate into the mucosal folds, which helps the drug to contact the lesion and pathogenic factors, and facilitates the drug absorption.

### DETAILED DESCRIPTION

Each component of the traditional Chinese medicine composition of the disclosure provides the following pharmacological effects:

In Traditional Chinese Medicine, Smilax Glabra Root is dried rhizome and root of Smilax Glabra that belongs to family Smilacaceae. Smilax Glabra Root tastes Sweet and belongs to the ‘Herbs that dispels Dampness and relieve Toxicity’ category. As suggested by the category Smilax Glabra Root is Neutral in nature and thought to target the Stomach and the Liver. “Neutral” means that Smilax Glabra Roots typically do not affect the balance in your body. The primary conditions or symptoms for which Smilax Glabra Root may be prescribed by TCM doctors to treat dysuria, leucorrhoea, carbuncle, scrofula, and scabies. Smilax glabra root mainly contains flavonoids, tannins, and resins, such as astilbin, isoengelitin, 3,5,4′-trihydroxystilbene, L-epicatechin, and succinic acid.

In Traditional Chinese Medicine, Cinnamon Twigs are dried young branches of Chinese Cinnamon that belongs to the family Lauraceae. Cinnamon Twigs taste Pungent and Sweet, and belong to the ‘Warm/Acrid herbs that release the Exterior’ category. As suggested by the category Cinnamon Twigs are plants that are Warm in nature and thought to target the Lung, the Heart, and the Bladder. “Warm” means that Cinnamon Twigs tend to help people who have too much “cold” in their body. The main actions of Cinnamon Twigs according to TCM: relieves the Exterior through sweating, disperse Cold and relieve Pain, removes obstruction of Yang and promotes the circulation of Yang Qi in the chest. The primary conditions or symptoms for which Cinnamon Twigs may be prescribed by TCM doctors: common cold, abdominal pain, dysmenorrhea, palpitations, edema, and phlegm fluid accumulation. Cinnamon Twigs contains volatile oils such as cinnamaldehyde and cinnamic acid, as well as phenols, organic acids, glycosides, coumarin and other ingredients.

In Traditional Chinese Medicine, Chinese Knotweed is the aerial part of Chinese Knotweed that belongs to the family Polygonaceae. The whole plant can be applied for medical use. Chinese Knotweed is Cold in nature and tastes bitter and sour. “Cold” means that Chinese Knotweed typically help people who have too much “heat” in their body. The main actions of Chinese Knotweed according to TCM includes: clear Heat and release Toxicity, drain Dampness, cool blood and stop itching, remove nebula and improve the visual acuity. The primary conditions or symptoms for which Chinese Knotweed may be prescribed by TCM doctors: dysentery, dyspepsia, hepatitis, diphtheria, whooping cough, corneal clouding, fungal vaginitis, leucorrhea, furunculosis, pustules in children, eczema, snake bites. Chinese Knotweed mainly contains indigo glycosides, polygonatum, p-coumaric acid, ferulic acid, vanillic acid, protocatechuic acid, caffeic acid, etc.

In Traditional Chinese Medicine, Commelina Communis is a perennial herbaceous plant that belongs to the family Commelinaceae. The aerial part can be applied for medical use. Commelina Communis is Cold in nature and tastes slightly bitter and sweet. The main actions of Chinese Knotweed according to TCM includes: clears Heat, releases Toxicity, and induces urination. The primary conditions or symptoms for Commelina Communis may be prescribed by TCM doctors: edema, tinea pedis, dysuria, cold, erysipelas, mumps, jaundice hepatitis, fever dysentery, malaria, epistaxis, hematuria, bleeding, leucorrhea, sore throat, carbuncle and furuncle. Commelina Communis mainly contains protocatechuic acid, caffeic acid, p-hydroxybenzoic acid, methyl gallate, coylidin-7-O-β-D-glucoside, isoquercetin, isovitexin, etc.

In the prescription of the disclosure, Smilax Glabra Root is a Jun (sovereign) ingredient drug used to target the disease or main symptoms suffered. Cinnamon Twigs and Chinese Knotweed are Chen (minister) ingredient drugs used to assistant Jun to dispels dampness, and relieve toxicity for detumescence. Commelina Communis is a Zuo (assistant) ingredient drug that help to relive and eliminate toxicities or side effects caused by Jun and Chen drugs. The traditional Chinese medicine composition of the disclosure protects females against diseases caused by HPV, such as genital warts, genital ulcer, problems urinating, as well as vaginal itching and pain.

Smilax glabra root contains two main active ingredients: flavonoids and tannins. Cinnamon Twig contains phenylpropanoids such as cortex aldehydes and cinnamic acid. Chinese Knotweed contains organic acids such as indoxyl sulfate. Commelina Communis contains organic acids and flavonoids. The above effective ingredients can be extracted from the raw materials using ethanol, while removing the macromolecules insoluble in ethanol and involved in allergic reactions, such as protein and glycoprotein. Therefore, the disclosure improves safety on active pharmaceutical ingredients extracted from the raw materials.

Optionally, the above effective ingredients can be extracted by dissolving the raw materials in water, and eluting the extract with the macroporous absorbent resin. The extraction method can also remove the above macromolecules involved in allergic reactions, enhance drug safety, provides a low-cost water extraction process with minimal impurity levels, thereby achieving a significantly greater therapeutic effect in a patient.

To further illustrate the disclosure, embodiments detailing a traditional Chinese medicine composition are described below. It should be noted that the following embodiments are intended to describe and not to limit the disclosure.

### Example 1

A traditional Chinese medicine composition for treatment of HPV infection comprises following raw materials: 40 parts by weight of smilax glabra root, 15 parts by weight of Cinnamon Twig, 10 parts by weight of Chinese Knotweed and 15 parts by weight of Commelina Communis.

The raw materials were mixed and pulverized into decoction pieces. The decoction pieces were dissolved in 60% ethanol, and the volume of the ethanol was 10 folds of the mass of the decoction pieces. The decoction pieces were decocted at 70° C. for 2 hours, filtered to give a filter residue for later use. A first filtrate was collected. The filter residue was decocted 3 times with 60% ethanol at 70° C., the volume of which was 10 folds of the mass of the filter residue, 2 hours for each time, and a second filtrate was obtained. The first filtrate and the second filtrate were mixed and concentrated at 60° C. under reduced pressure to give the pharmaceutical cream with a relative density of 1.10-1.40.

Preparation of gel dosage form: A mixture of polyhexamethylene biguanide and carbomer was soaked in purified water for 4 hours, thereby forming a gel matrix for later use. To the gel matrix, the pharmaceutical cream, glycerin and azone were added, and then purified water was added. Triethanolamine was added dropwise to the gel matrix to adjust pH=7, and stirred to give a gel dosage form.

The pharmaceutical cream is not limited to the gel dosage form. A suppository or other dosage forms can be prepared by mixing glycerin and an excipient to a suppository mold.

### Example 2

Example 2 is basically the same as Example 1 except that the traditional Chinese medicine composition comprises following raw materials: 30 parts by weight of smilax glabra root, 15 parts by weight of Cinnamon Twig, 10 parts by weight of Chinese Knotweed and 15 parts by weight of Commelina Communis.

The raw materials were mixed and pulverized into decoction pieces. The decoction pieces were dissolved in 70% ethanol, and the volume of the ethanol was 10 folds of the mass of the decoction pieces. The decoction pieces were decocted at 75° C. for 1.5 hours, filtered to give a filter residue for later use. A first filtrate was collected. The filter residue was decocted 4 times with 75% ethanol at 70° C., the volume of which was 10 folds of the mass of the filter residue, 1.5 hours for each time, and a second filtrate was obtained. The first filtrate and the second filtrate were mixed and concentrated at 60° C. under reduced pressure to give the pharmaceutical cream with a relative density of 1.35.

An excipient was added to the pharmaceutical cream to prepare a suppository. Specifically, to 10 g of the pharmaceutical cream, the excipient containing 60 g of glycerin and 55 g of polyethylene glycol was added. The mixture was melted, poured into a suppository mold, cooled, removed from the mold and packed individually to obtain a suppository.

### Example 3

A traditional Chinese medicine composition comprises following raw materials: 55 parts by weight of smilax glabra root, 13 parts by weight of Cinnamon Twig, 15 parts by weight of Chinese Knotweed and 18 parts by weight of Commelina Communis.

The raw materials were mixed and pulverized into decoction pieces. The decoction pieces were dissolved in 60% ethanol, and the volume of the ethanol was 8 folds of the mass of the decoction pieces. The decoction pieces were decocted at 55° C. for 4 hours, filtered to give a filter residue for later use. A first filtrate was collected. The filter residue was decocted 5 times with 60% ethanol at 55° C., the volume of which was 8 folds of the mass of the filter residue, 3 hours for each time, and a second filtrate was obtained. The first filtrate and the second filtrate were mixed and concentrated at 60° C. under reduced pressure to give the pharmaceutical cream with a relative density of 1.3.

To 100 g of the pharmaceutical cream, 1 L of water, 30 g of polysorbate-80, and 5 g of sodium benzoate were added and mixed. The mixture was packed individually to obtain a lotion.

### Example 4

A traditional Chinese medicine composition comprises following raw materials: 50 parts by weight of smilax glabra root, 13 parts by weight of Cinnamon Twig, 18 parts by weight of Chinese Knotweed and 12 parts by weight of Commelina Communis.

The raw materials were mixed and pulverized into decoction pieces. The decoction pieces were dissolved in 60% ethanol, and the volume of the ethanol was 8 folds of the mass of the decoction pieces. The decoction pieces were decocted at 55° C. for 4 hours, filtered to give a filter residue for later use. A first filtrate was collected. The filter residue was decocted 5 times with 60% ethanol at 55° C., the volume of which was 8 folds of the mass of the filter residue, 3 hours for each time, and a second filtrate was obtained. The first filtrate and the second filtrate were mixed and concentrated at 60° C. under reduced pressure to give the pharmaceutical cream with a relative density of 1.2.

To the gel matrix, a mixed solution containing 150 g of the pharmaceutical cream and 50 g of glycerol, 20 g of azone was added. Thereafter, purified water was added, and triethanolamine was added dropwise to the gel matrix to adjust pH=7. The mixture was stirred and packed individually to give a gel dosage form.

### Example 5

A traditional Chinese medicine composition for treatment of HPV infection comprises following raw materials: 30 parts by weight of smilax glabra root, 15 parts by weight of Cinnamon Twig, 15 parts by weight of Chinese Knotweed and 15 parts by weight of Commelina Communis.

The raw materials were mixed, pulverized into decoction pieces, and dissolved in 60% ethanol, and the volume of the ethanol was 10 folds of the mass of the decoction pieces. The decoction pieces were filtered to give a filter residue for later use. A first filtrate was collected. The residue was dissolved in 60% ethanol, the volume of which was 10 folds of the mass of the filter residue, filtered to yield a second filtrate. The filtrates were obtained 3 times through reflux extraction, 2 hours for each time. The first filtrate and the second filtrate were mixed and concentrated at 60° C. under reduced pressure to give the pharmaceutical cream with a relative density of 1.2.

1 L of water, 50 g of polysorbate-80, and 5 g of sodium benzoate were added to 100 g of the pharmaceutical cream and mixed. The mixture was packed individually to obtain a lotion.

### Example 6

A traditional Chinese medicine composition for treatment of HPV infection comprises following raw materials: 30 parts by weight of smilax glabra root, 10 parts by weight of Cinnamon Twig, 10 parts by weight of Chinese Knotweed and 10 parts by weight of Commelina Communis.

The raw materials were mixed and pulverized into decoction pieces. The decoction pieces were dissolved in water, the volume of which was 12 folds of the mass of the decoction pieces, filtered to give a filter residue for later use. A first filtrate was collected. The filter residue was extracted 2 times through reflux extraction, 2 hours for each time, to yield a second filtrate. The first filtrate and the second filtrate were mixed, absorbed using an AB-8 macroporous absorbent resin, and eluted consecutively with water and 75% ethanol. The eluent comprising 75% ethanol was collected and concentrated at 60° C. under reduced pressure to give the pharmaceutical cream with a relative density of 1.2.

5 g of dried pharmaceutical cream at 60° C. was mixed with an excipient containing 70 g of glycerin and 50 g of polyethylene glycol. The mixture was melted, poured into a suppository mold, cooled, removed from the mold and packed individually to obtain a suppository.

### Example 7

A traditional Chinese medicine composition for treatment of HPV infection comprises following raw materials: 45 parts by weight of smilax glabra root, 10 parts by weight of Cinnamon Twig, 15 parts by weight of Chinese Knotweed and 10 parts by weight of Commelina Communis.

The raw materials were mixed and pulverized into decoction pieces. The decoction pieces were dissolved in 60% ethanol, the volume of which was 10 folds of the mass of the decoction pieces, and filtered to give the filtrate. The filtrates were obtained 2 times through reflux extraction, 2 hours for each time. The filtrates were mixed, absorbed using the AB-8 macroporous absorbent resin, and eluted consecutively with water and 75% ethanol. The eluent comprising 75% ethanol was collected and concentrated at 60° C. under reduced pressure to give the pharmaceutical cream with a relative density of 1.3.

Preparation of gel dosage form: 500 mL of purified water was added to a mixture of 15 g of polyhexamethylene biguanide and 3 g of carbomer. The mixture was soaked in water for 4 hours, thereby forming a gel matrix for later use. To the gel matrix, a mixed solution containing 150 g of the pharmaceutical cream, 50 g of glycerin, and 10 g of azone was added, followed by addition of 1000 mL of purified water. Triethanolamine was added dropwise to the gel matrix to adjust pH=7, and stirred to give a gel dosage form.

### Example 8

The Traditional Chinese Medicine Composition Inhibits the Proliferation of Hela Cells

The percentage inhibition on HeLa cell proliferation for each compound prepared in Examples 1-7 was determined by MTT assay (MTT Cell Proliferation and Cytotoxicity Assay Kit). HeLa cells was cultured in a 96-well plate for 6.0×10³ cells per well, and allowed to adhere to the surface of the 96-well plate. Serum-free medium was added to each well of control group. Cidofovir was diluted in purified water such that the final concentration of cidofovir in the assay was 10 μmol/mL, and the resulting solution was added to each well of positive control group. Each compound of Examples 1-7 was diluted in water to a crude drug concentration of 25 g/L, and the resulting solution was added to the 96-well plate for a final volume of 200 μL per well. 200 μL of complete medium was only added to each well of blank control group. Each experimental group was done in 6 well replicates. After incubation of 24, 48, and 72 hours, 20 μL of 5 g/L MTT was added to each well and the plates were further incubated at 37° C. for 4 hours. Following discarding the supernatant, 200 μL of dimethyl sulfoxide (DMSO) was added to each well and the plates were shaken sufficiently as so to dissolve the crystals. Absorbance (A) of the wells at 490 nm was measured. These measurements then are used to calculate the percentage inhibition of cell growth: percentage inhibition=1−(T_{A}−C_{A})×100%, where T_{A} refers to the average absorbance of treatment group, and C_{A} refers to the average absorbance of control group. Each experiment was performed in triplicate, and the average percentage inhibition in each group are shown in Table 1.

TABLE 1
Comparison of inhibition percentage of pharmaceutical
preparations on HeLa cell proliferation

| Group | 24 hours | 48 hours | 72 hours |
|---|---|---|---|
| Blank group | 1.56 ± 0.44 | 3.20 ± 1.01 | 4.05 ± 0.52 |
| Cidofovir | 32.24 ± 2.08 | 48.58 ± 3.50 | 58.09 ± 3.21 |
| Example 1 | 42.74 ± 1.48 | 55.83 ± 2.47 | 74.89 ± 2.63 |
| Example 2 | 41.45 ± 1.94 | 54.73 ± 2.91 | 74.75 ± 2.62 |
| Example 3 | 43.82 ± 2.78 | 57.46 ± 2.18 | 77.35 ± 3.11 |
| Example 4 | 41.32 ± 2.03 | 57.74 ± 2.95 | 76.48 ± 2.27 |
| Example 5 | 39.55 ± 2.17 | 56.08 ± 3.24 | 75.84 ± 2.58 |
| Example 6 | 42.17 ± 2.66 | 58.24 ± 1.97 | 78.01 ± 3.06 |
| Example 7 | 43.58 ± 3.04 | 60.62 ± 2.65 | 80.33 ± 3.14 |

The traditional Chinese medicine composition of the disclosure for use in treatment of HPV infection has the ability to inhibit cell proliferation in the HeLa cells. Percentage inhibition increases with the increase of culture time, which indicates an effect that is statistically significant compared to the corresponding control group.

### Example 9

Evaluation of the Efficacy of the Compounds in Treatment of HPV Infection

Compounds prepared in Examples 5-7 was tested and evaluated for efficacy. The volunteers infected with high-risk HPV were randomly divided into equal groups, and each group contained 50 people. A suppository comprising a blank matrix was delivered to the cervix of each volunteer in blank control group. A suppository comprising an interferon was delivered to the cervix of each volunteer in positive control group. The lotion prepared in Example 5 was used to clean the area surrounding the labia and inside the vagina. The suppository prepared in Example 6 and the gel dosage form prepared in Example 7 were delivered to the cervix, respectively. After 12 weeks of drug treatment, each volunteer got a physical examination involving recording the symptoms that the volunteers feel subjectively, gynecological examination, and microscopic examination of vaginal discharge, so as to analysis and evaluate the efficacy of the compounds in treatment of patient with high-risk HPV. A negative HPV test means that the volunteer is cured. A reduction in positive viral load greater than (or equal to 50%) is considered to be effective (Digene HC2 HPV DNA Test), otherwise, the drug is invalid against HPV. The results are shown in Table 2 below.

TABLE 2
Comparison of clinical efficacy of
pharmaceutical preparations on HPV

| | | | | | Total effective |
|---|---|---|---|---|---|
| Group | Cases | Cured | effective | invalid | rate/% |
| Blank control | 50 | 0 | 0 | 50 | 0 |
| group | | | | | |
| Positive | 50 | 6 | 30 | 14 | 72 |
| control group | | | | | |
| Example 5 | 50 | 34 | 9 | 7 | 86 |
| Example 6 | 50 | 39 | 6 | 5 | 90 |
| Example 7 | 50 | 38 | 8 | 4 | 92 |

Result of Table 2 show that the total effective rate of the traditional Chinese medicine composition in treatment of high-risk HPV infection is high than 86%, in which the cure rate is higher than 68%.

During the 6-month experiment period, by building an international standardized comparison model and performing a series of experiments for pharmacological and pathological tests, the traditional Chinese medicine composition of the disclosure provides superior capability for identification and inactivation of HPV in the cells, and has ability to kill the host HeLa cell of cervical cancer caused by HPV, especially the high-risk mucosal HPV types.

### Example 10

Clinical Case I

A 32-year-old female was diagnosed with HPV33 in May 2016 and failed to respond to treatment with interferon. Patient still tested positive for HPV33 in October 2017 and was coinfected with HPV18, 52, and 56. Colposcopy and directed biopsies (CDB) was performed and showed CIN1. From December 2017 to March 2018, the lotion prepared in Example 5 was used for 5 consecutive days followed by 2 consecutive days of rest, for nine seven-day cycles. Note no treatment during menstrual periods. Then HPV tested negative.

### Example 11

Clinical Case II

A 62-year-old female was diagnosed with HPV58 in September 2011 and the infection had persisted for more than 6 years. Patient still tested positive for HPV58 in September 2017 and was coinfected with HPV81. From December 2017 to March 2018, the suppository prepared in Example 6 was used for 5 consecutive days followed by 2 consecutive days of rest, for nine seven-day cycles. Note no treatment during menstrual periods. The patient was tested negative for HPV43, 51, and 83 in September 2018.

### Example 12

Clinical Case III

A 59-year-old female was diagnosed with HPV for half a year after 10 years after hysterectomy. Patient failed to respond to treatment with a gel dosage form obtained from a TCM company. From June 2018 to August 2018, the gel dosage form prepared in Example 7 of the disclosure was used for 5 consecutive days followed by 2 consecutive days of rest, for nine seven-day cycles. Note no treatment during menstrual periods. HPV43, 51, and 83 tested negative in November 2018.

In conclusion, the traditional Chinese medicine composition of the disclosure has the ability to kill HPV and human cervical cancer HeLa cells, and to treat cervicitis, which is suitable for treatment of HPV infection and the associated diseases, such as vaginitis, cervicitis, and cervical ectropion. The TCM composition has a mild smell safe for human body and acts with mil side effects, which can directly act on the vagina without irritation. The one-time bolus injection of gel dosage form, suppository and lotion dosage form are easy for patients to use and improve patient compliance. The drugs in above dosage form can remain in the vagina for a long time and penetrate into the mucosal folds, which helps the drug to contact the lesion and pathogenic factors, and facilitates the drug absorption.

It will be obvious to those skilled in the art that changes and modifications may be made, and therefore, the aim in the appended claims is to cover all such changes and modifications.

What is claimed is:

1. A pharmaceutical composition, comprising:
30-60 parts by weight of Smilax Glabra Root;
10-20 parts by weight of Cinnamon Twig;
10-20 parts by weight of Chinese Knotweed; and
10-20 parts by weight of Commelina Communis.

2. The pharmaceutical composition of claim 1, comprising:
30-55 parts by weight of Smilax Glabra Root;
12-18 parts by weight of Cinnamon Twig;
10-18 parts by weight of Chinese Knotweed; and
12-18 parts by weight of Commelina Communis.

3. A pharmaceutical preparation, comprising the pharmaceutical composition of claim 1.

4. The pharmaceutical preparation of claim 3, having a dosage form of creams, lotions, gels, or suppositories.

5. A method of preparing the pharmaceutical preparation of claim 3, the method comprising:
1) mixing 30-60 parts by weight of Smilax Glabra Root, 10-20 parts by weight of Cinnamon Twig, 10-20 parts by weight of Chinese Knotweed, and 10-20 parts by weight of Commelina Communis, thus yielding a mixture;
2) dissolving the mixture in a solvent, decocting the mixture dissolved in the solvent, thus yielding a first filtrate and a first residue;
3) collecting the first filtrate, dissolving the first residue in the solvent, and decocting the first residue dissolved in the solvent, thus yielding a second filtrate and a second residue;
4) repeating 3), mixing collected filtrates, and condensing the filtrates to yield an extract having a relative density of 1.10-1.40 at 60° C.

6. The method of claim 5, wherein the solvent is an ethanol solution; the ethanol solution has a concentration of 50-80% (v/v), and a volume weight ratio of the ethanol solution to the mixture is 5-15.

7. The method of claim 5, wherein the solvent is water.

8. The method of claim 5, wherein in 2), the mixture and the filtrates are decocted in the solvent at 50-75° C. for 2-5 hours.

9. The method of claim 5, wherein in 4), repeating 3) for 2-5 times, and the filtrates are condensed at 50-80° C.

10. The method of claim 5, further adding an excipient to the extract to yield a gel or suppository.

11. The method of claim 5, further adding an excipient to the extract to yield a lotion.

12. A method for prevention and/or treatment of human papillomavirus (HPV) infection comprising administering a patient in need thereof the pharmaceutical preparation of claim 3.

13. A method for prevention and/or treatment of vaginitis, cervicitis, or cervical ectropion comprising administering a patient in need thereof the pharmaceutical preparation of claim 3.
